(19) United States
(12) Patent Application Publication
(10) Pub. No.: US 2014/0207046 A1
(43) Pub. Date: Jul. 24, 2014

(54) LED induced tattoo removal system and method of use

(71) Applicant: Stephen Feldman, New York, NY (US)

(72) Inventor: Stephen Feldman, New York, NY (US)

(73) Assignee: InrexRem

(21) Appl. No.: 13/694,223

(22) Filed: Jan. 24, 2013

(51) Int. Cl. A61N 5/06; A61M 35/00

(52) U.S. Cl. CPC A61N 5/0616; A61M 35/00
USPC 604/20; 606/9

(57) ABSTRACT

An LED induced tattoo removal system and method of use that utilizes an ultra bright LED having a principal peak with a peak at 640 nm-700 nm for tattoo removal without causing collateral skin damage and without causing pain to a subject is disclosed. The tattoo removal method includes an LED, which generates light in a specific range of wavelengths proven to be effective for this purpose. The method also includes optionally applying a vasodilator after the LED exposure and further includes optionally utilizing an electrical or chemical heat source after the LED exposure. The tattoo removal method comprises irradiation of the tattooed region using ultra bright LED light for a timed interval and the optional application of a vasodilator formulation, which increases blood circulation to the tattooed skin region and also increases the concentration macrophage cells in the treated skin region to metabolize the tattoo ink and its by-products.

### CROSS-REFERENCE TO RELATED APPLICATION

This patent application is a continuation-in-part of U.S. patent application Ser. No. 12/381,134 filed Mar. 6, 2009, by inventor, Frank Pellegrini, for an invention entitled, “Ultra Bright LED Induced Tattoo Removal”, and claims the benefit thereof under 35 U.S.C. 120 and claims the benefit of provisional application 61/068,369 and Ser. No. 12/381,134.

### BACKGROUND OF THE INVENTION

1. Field of the Invention

This invention relates generally to a tattoo removal system and, more particularly, to an LED (i.e. Light Emitting Diode) induced tattoo removal system and method of use that may include use of a vasodilator or heating for removing tattoos without causing collateral skin damage and with minimal pain to a subject with use of certain defined wavelengths in the red, orange-warm LED light spectrum.

2. Description of the Related Prior Art

A variety of systems and methods to remove tattoos are known in the prior art. In co-pending parent application Ser. No. 12/381,134, Applicant has previously disclosed U.S. Pat. No. 6,676,655, which utilizes LED pulsing light in the near IR region to treat various dermatological conditions, and US Patent Publication No. 2005/0148567, which describes a photosensitizer therapy method that may affect tattoo inks.

Further, prior art patent references including U.S. 2005/0131497 to Suzuki, US 2004/0030325 to Cahir et al., U.S. Pat. No. 6,936,044 to McDaniel and 2003/0004499 to McDaniel and U.S. 2007/0093798 to DeBenedictis et al., and U.S. Pat. No. 6,149,644 have been cited by or to Patent Office and made of record in the parent application of this case and U.S. Pat. No. 6,149,644.

However, the prior art systems and methods have many disadvantages. First, prior systems relied on cell disruption, the direct physical destruction of the cells in the tattooed region, removing ink from the cells and the surrounding area. These systems and methods are therefore known to overheat the epidermal skin layer adjacent the tattoo to be removed and to cause pain and possible scarring during treatment. The excess heat generated can also be transmitted to deeper skin tissues of the dermis, especially those containing hair and sweat glands.

Second, the prior art systems and methods for tattoo removal typically involve use of monochromatic light with relatively short duty cycles, such as lasers, that may not be absorbed by tattoo dyes of various colors. Third, many of the prior art methods for tattoo removal involve cosmetic surgery and, thus, are not affordable to those in need of treatment. Fourth, the prior art systems for tattoo removal cannot treat large skin surface areas and so the treatment is focused on a very small area of a tattoo. Fifth, the known systems of tattoo removal require specialized training of personnel who administer treatment to ensure that accepted therapy and practice are followed. Lastly, because the damaged tissue needs time to heal, many prior art treatments for tattoo removal cannot be repeated on a daily basis or at short intervals and, thus, progress is often very slow with little desire for the person having the tattoo to continue the treatment.

All of the hereinabove disadvantages are addressed if the LED device of this invention is utilized. The LED induced tattoo removal system of this invention has distinct advantages. Initially, LEDs have a significantly longer duty cycle with non pulsing and provide continuous energy output that has an advantageous light spectrum range to overcome drawbacks associated with prior art methods and devices.

Thus, there is a need for a tattoo removal system and method that uses relatively low-temperature light sources, such as ultra bright LEDs, which can treat large skin areas. Further, there is need for a tattoo removal system that uses the singular properties of continuous LED light irradiation, with a primary peak in the red, orange red-warm range of the visible spectrum about 640 nm-700 nm which penetrates sufficient depth through the outer skin without causing damage and which breaks down tattoo dye molecules by increasing molecular motion and bond deformation.

All these needs are addressed by the present invention which proposes an LED induced tattoo removal system and method of use that provides safe and effective treatment without expensive equipment, harsh chemical formulations, or the services of a highly trained healthcare professional or physician.

The tattoo removal system and method of the present invention provides effective tattoo removal by continuous irradiation (i.e. without pulsing) of the tattooed skin region using the light energy generated by ultra bright LEDs having a power of about 50 W and the optional application on the tattooed skin region of a vasodilator, such as a cream formulation containing L-arginine or a chemical or electrical heating source, such as a small commercial heating pad, or a commercial heat pack that generates heat by the mixing of two chemicals. The optional use of a vasodilator cream or heat after application of the LED treatment may lead to the increased infiltration of macrophage cells to the treated region, which metabolize the tattoo ink and its by-products.

### SUMMARY OF THE INVENTION

The tattoo removal system and method of the present invention includes irradiating the tattooed skin region with continuous, ultra bright LED light energy of predetermined wavelengths in red-orange or red wavelengths around 640-700 nm. The present tattoo removal system and method may include the application of a vasodilator or heat on the tattooed skin region for increasing blood circulation and increasing the concentration of macrophage cells to the treated skin area to metabolize the tattoo ink and its by-products.

The present method for tattoo removal includes the steps of positioning an optical device including ultra bright LEDs of predetermined wavelengths at a specific efficacious distance from the subject tattoo to generate heat within the tattoo dyes by irradiating the subject tattoo with continuous LED energy for a specific period of time.

### BRIEF DESCRIPTION OF THE DRAWINGS

The above mentioned and other features, aspects and advantages of the present invention will become better understood with regard to following description, appended claims and accompanying drawings:

FIG. 1 shows the approximate spectrum of the LED of the invention;

FIGS. 2-3 is diagram showing a tattoo removal system for use with the present invention;

FIG. 4 is initial and final photographs respectively of a tattooed region of a male Wistar rat exposed to LED irradiation once a day for 30 minutes with the LED.

FIG. 5 is initial and final photographs respectively of black tattooed region from a male Wistar rat exposed to LED irradiation once a day for 60 minutes;

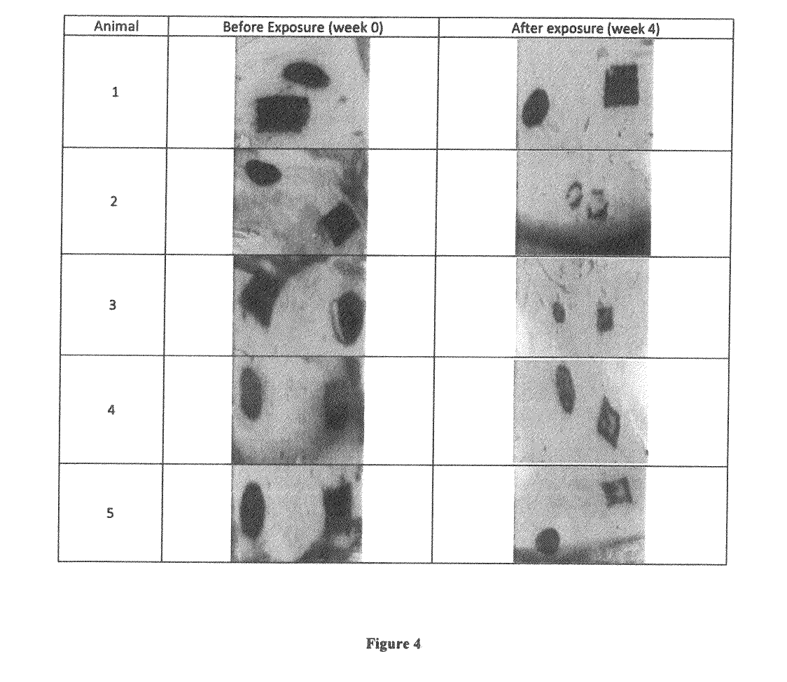

FIG. 6 is initial and final photographs respectively of a black tattooed region from a male Wistar rat exposed to LED irradiation once a day for 30 minutes with a vasodilator applied after the LED irradiation

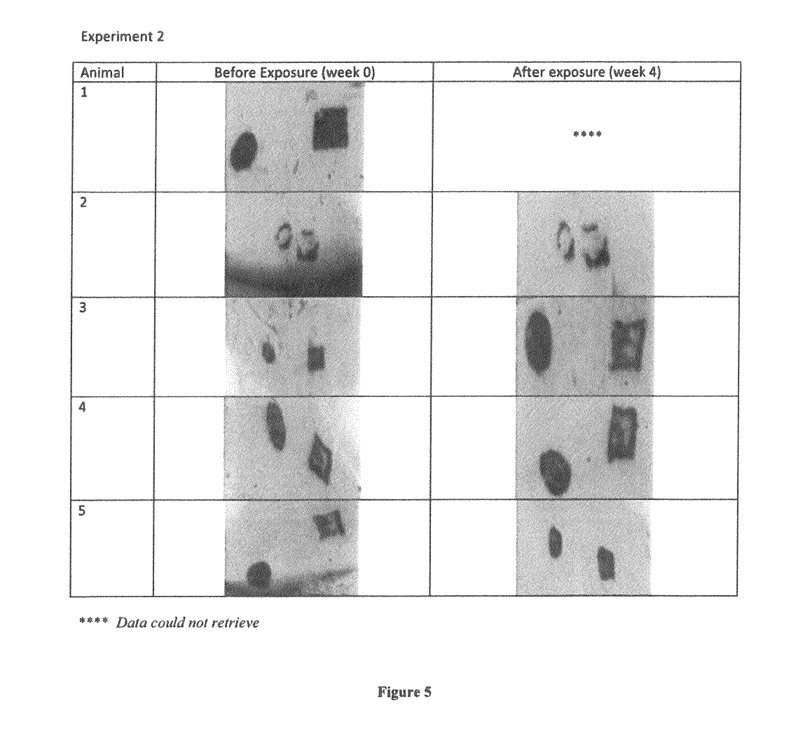

FIG. 7 is initial and final photographs respectively of black tattooed region of a male Wistar rat exposed to LED irradiation once a day for 60 minutes with vasodilator cream; applied immediately after the LED irradiation.

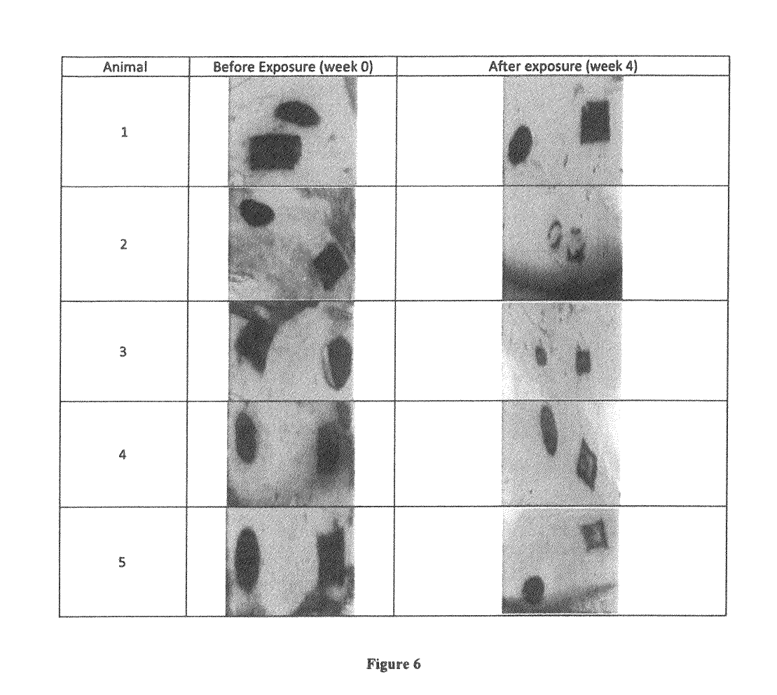

FIGS. 8A-8D are initial and final photographs respectively of tattooed region of a male Wistar rate exposed to LED irradiation.

FIGS. 9A-9B are initial and final photographs respectively of blue tattooed region from a male Wistar rat exposed to LED irradiation once a day for 60 minutes followed by application of vasodilator cream.

FIGS. 10A-10B are initial and final photographs respectively of black tatttoed region from a male Wistar rat exposed to LED irradiation once a day for 60 minutes followed by application of L-Arginine cream.

FIGS. 11A-11B are initial and final photographs respectively of blue tattooed region from a male Wistar rat exposed to LED irradiation once a day for 60 minutes followed by application of L-Arginine cream.

FIG. 12 are initial and final photographs respectively of black tattooed region from a male Wistar rat exposed to LED irradiation twice a day for 60 minutes with a application of vasodilator cream.

FIG. 13 are initial and final photographs respectively of blue tattooed region from a male Wistar rat exposed to LED irradiation twice a day for 60 minutes with a application of vasodilator cream.

### DETAILED DESCRIPTION OF THE INVENTION

Although specific terms are used in the following description for sake of clarity, these terms are intended to refer only to particular description, and are not intended to define or limit the scope of the invention.

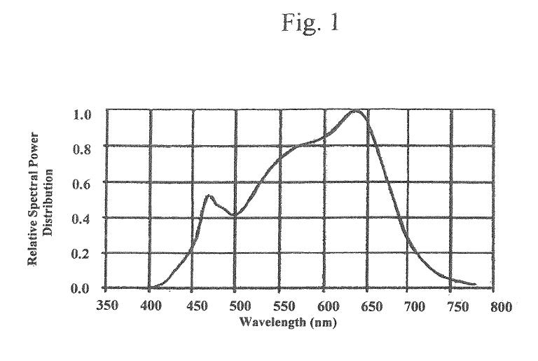

FIG. 1 shows the approximate spectrum of the LED of the present invention.

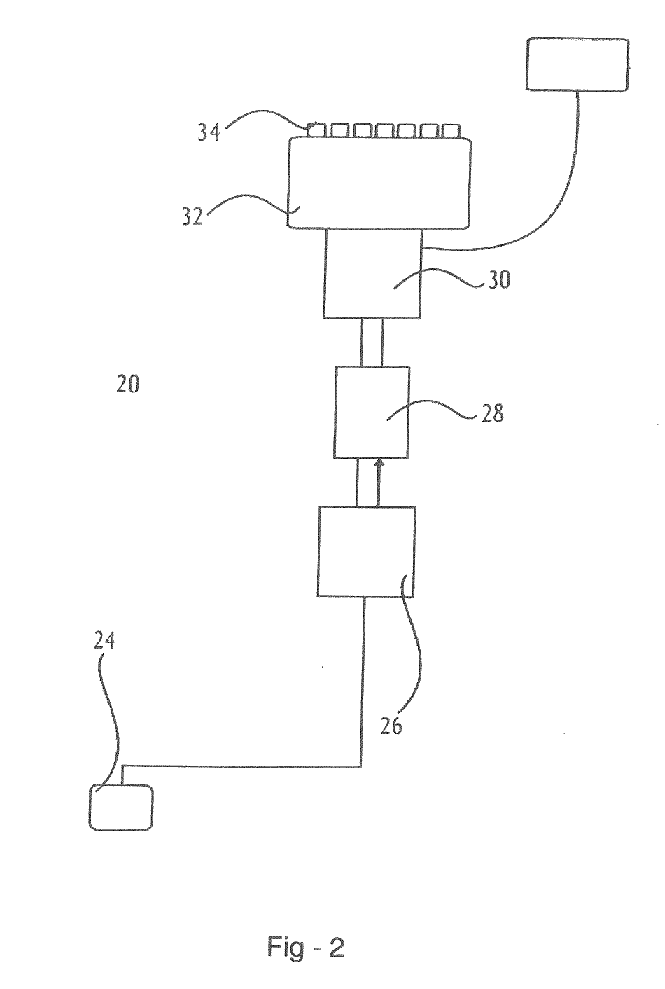

FIG. 2 is a diagram that depicts a system for tattoo removal, indicated generally at 20. The components of the present system 20 include an AC power supply that supplies power to an AC to DC converter which, in turn, is electrically connected to a timer, a Printed Circuit Board (hereinafter PCB 30) and an LED 44 for irradiating a tattooed skin region.

The warm LED spectrum shown in FIG. 1 has a principal peak at about 640 nm with a partial peak at about 550 nm and a smaller peak at about 470 nm. The principal peak allows for substantial radiation in the red, orange, region of the spectrum.

The period of time during which the LED is used to irradiate a tattooed skin region varies but is usually at least 5 minutes.

Still referring to FIG. 2, the LED 44 is configured such that the LED may be properly positioned to direct the LED energy at a desired angular orientation to the subject tattoo. For example, in the embodiment shown in FIG. 2-3, is designed to target a tattoo on a person. Of course, alternative configurations and the LED are considered to be within the scope of the present invention. Thus, the embodiment of the present invention shown in FIG. 2-3 is considered merely illustrative and not restrictive in any sense. The most efficacious distance from the LED to the tattoo is a distance where the LED light will shine on the tattoo and lead to its lessening or removal.

### Example 1

The operator places the apparatus approximately 1 to 2 inches to 10 cm above the tattooed. The apparatus contains an LED of 50 W with light in the 640 nm-700 nm region as shown in FIG. 1. The tattoo area is then exposed to the continuous light for 15 minutes. During this period of time, the light penetrates into the dermal layer in which the tattoo resides. The absorption of the energy by the tattoo ink results in heat generated in the ink molecules to result in the tattoo fading or being removed.

### Example 2

The operator places the apparatus approximately 1 to 2 inches above the tattooed area. The apparatus contains an LED of 50 W or 3000 joules per minute in the 640 nm-700 nm region of the spectrum. The tattoo area is then exposed to the continuous light generated by the LED for 15 minutes. During this period of time, the light penetrates through the epidermis and into the dermal layer in which the tattoo resides. The absorption of the energy by the tattoo ink results in heat generated in the ink molecules A thin layer of 10% to 15% of L-Arginine is applied directly to the tattoo area by the operator. This, results in the tattoo fading or being removed.

### In Operation

The energy contained in the light beam is absorbed by the tattoo ink dyes. This absorbed energy will result in an increased stretching, vibration and bending of the bonds which hold the dye (ink) molecules together. Ultimately, these bond stresses cause bond deformation with resulting bond failure. The frequencies chosen are those which produce energies which are absorbed by the bonds in the dyes, but have minimal absorption by melanin in the skin or hemoglobin in the blood. Melanin and hemoglobin have maximum absorptions below 600 nm. Maximum absorption for melanin is 335 nm and for hemoglobin 310 nm.

For the light produced to be beneficial for tattoo removal, ultra bright LED's with high enough energy output are used. The output energy, such as, for a red color ultra bright LED (640-700 nm), will be about 88 joules per square inch. The brightness of light depends upon its photon density. The brighter the light, the greater will be its photon density. Since each photon at a given wavelength has the same energy, the greater the photon density, the greater will be the energy content of the light. Thus, using ultra bright LED's leads to a higher level of energy in the light beam. At 640 to 770 nm wavelength there is no light absorption by either melanin (skin coloring agent) or hemoglobin in blood. The greater the light intensity, the greater will be the energy content of the light. Heat is generated when the light is absorbed by the molecules thus increasing the molecular motion.

Melanin and hemoglobin do not absorb well at 640 to 700 nm of wavelength. Light does not generate heat on the skin at 640 to 700 nm of wavelength since the main chromophore of the skin is melanin but will generate heat on tattoo dyes since these dyes absorb energy in the 640-700 nm range of wavelength. Therefore, little to no heat is generated on the surface of the skin, but the light penetrates through the epidermis into the dermis where the tattoo ink resides. These frequencies are absorbed by dye molecules causing increasing molecular motion and bond deformation. Ultra bright LEDs are from approximately 50 to 500 times brighter than standard LEDs. Thus, their energy content is likewise 50 to 500 times greater. The frequency of light used to destabilize the bonds in tattoo inks depends upon the composition of the ink and its color.

The method is able to work for removing tattoos by the energy contained in the light beam being absorbed by the tattoo ink dyes. This absorbed energy will result in an increased stretching, vibration and bending of the bonds which hold the dye (ink) molecules together. Ultimately, these bond stresses cause bond deformation with resulting bond failure. The output energy, such as, for a red color ultra bright LED. The proximity of the bulbs and the amount of energy emitted into the tattoo will penetrate the epidermis and into the dermis in which the tattoo is situated.

In another embodiment, Arginine cream can be applied to the tattooed region after the LED treatment. It creates enlarged blood vessels which bring greater blood flow to the tattoo area. In addition, it creates an increase in the immune system response. These mechanisms help speed up the removal of the by-products of the degradation of the tattoo dyes, thus, allowing for the tattoo to fade more quickly.

In one embodiment, an IRM (immune response modifier) compound can be applied. Specifically, IRM compounds containing Arginine can also increase the concentration of macrophages in the blood. Macrophages are specifically located in the lymph nodes and are white blood cells that phagocytize necrotic cell debris and foreign material, including viruses, bacteria, and tattoo ink.

The IRM compound may be selected from a group consisting of imidazoquinoline amine; a tetrahydroimidazoquinoline amine; an imidazopyridine amine; a 1,2-bridged imidazoquinoline amine; a 6,7-fused cycloalkylimidazopyridine amine; animidazonaphthyridine amine; a tetrahydronaphthyridine amine; an oxazoloquinoline amine; a thiazoloquinoline amine; an oxazolopyridine amine; a thiazolopyridine amine; an oxazolonaphthyridine amine; a thiazolonaphthyridine amine; or a IH-imidazodirner fused to a pyridine amine, a quinoline amine, a tetrahydroquinoline amine, a naphthyridine amine, and a tetrahydronaphthyridine amine.

### Laboratory Animal Studies

An investigation to evaluate the tattoo removal potential of the described process utilizing the LED light source was undertaken using Wistar rats as a laboratory animal model. The Wistar rat is currently one of the most popular rat strains used for laboratory research. The tattoos created on the animal's skin did not automatically fade without treatment and also did not cause any apparent dermatological toxic effects. Histopathological investigation of all the studies revealed that the LED exposure on the tattooed region did not cause any ulceration, inflammation, congestion or fibrosis.

The LED 42 used throughout the described experiments is commercially available and sold under the tradename, Edi Star Series from Edison Opto, Warm LED Part Number ENSX-05-0707-EE-1, Luminous Flux (Im) (at 1_{F}=2400 mA/3000 mA and T,=25° C. 2800, Forward Votage (V) (at 2400 mA/3000 mA and T, =250) 24.5. The test current utilized was 350 mA (i.e. milliamperes) and the drive current was 700 mA. The minimum color temperature was in the 2540K to 4500K range, the maximum color temperature was in the 3500K to 10000 k range and the typical color temperature was in the 3100K to 6500K range. The total Typical CRI (Color Rendering Index) ranged from 70 to 85 for these experiments. The LEDs have the spectrum of FIG. 1, with a principal peak at about 640. A single LED or an array of LEDs can be used.

Twenty male Wistar rats were procured for these studies. After an acclimatization period of one week the hair on the dorsal side of each animal was removed using a commercially available hair removal cream. Throughout the series of experiments described herein, animals were grouped in red, blue, black and green color tattoo groups. However, not every study included all of these color groups.

The rats were tattooed on their dorsal (i.e. back) sides under anesthesia (i.e. ketamine (80 mg per kg intraperitoneal) and xylazine (8 mg per kg intraperitoneal) by an experienced tattoo applier. The tattoos were created using commercially available inks sold under the trade name, Kaplan East Coast Tattoo Supply ink.

In the first four studies two tattoo designs were made on each animal. One was circular in shape and the other was square. After making the tattoos a quarantine period of one week was observed to see whether the tattoo would naturally disappear.

The following procedure was followed for the first four weeks after the quarantine period:
Each day the tattooed skin of each animal was exposed to continuous LED energy for 30 minutes. For specified subjects, vasodilator cream was applied on both the square and the circular tattoo after exposure.
   The following protocol was observed for the subsequent four weeks:
Each day the tattooed skin of each animal was irradiated with continuous LED energy for 60 minutes. The vasodilator cream was applied on both the square and the circular tattoo after exposure.

During the entire experiment, the animals were restrained in proper position using a restrainer to ensure proper orientation to the LED light. The distance between the LED 42 and the tattooed skin was kept at approximately ten centimeters. The LED 42 was adjusted vertically above the tattooed skin region.

Each week excess hair was removed by hair removing cream. The tattoo on each animal was scanned at 250 DPI (dots per inch) using a CCD (charge-coupled device) scanner and the pictures saved in JPEG format to calculate the average density of the tattoo.

### Example 1

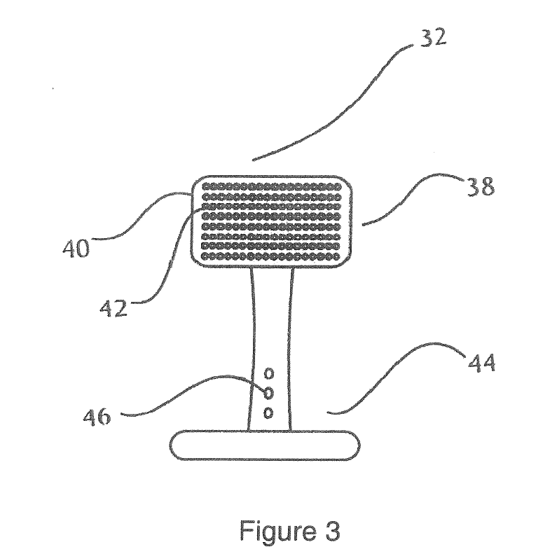

In this example, five rats were prepared with a circular and square tattoo. The tattoo was created using a commercially available back ink sold under the tradename, Kaplan East Coast Tattoo Supply ink. After the completion of a quarantine period, LED exposure was initiated on the subject tattoo. For a period of four weeks, the tattooed skin area was exposed to continuous LED light generated by ultra bright white LEDs for thirty minutes every day with the results detailed in Table 1: below and are shown in FIG. 4.

TABLE 1
Fading pattern of the circular tattoo (Exposure period: 30 minutes)

| | Average | | Average | Average | Average |
|---|---|---|---|---|---|
| | color | Average color | color | color | color |
| | intensity | intensity | intensity | intensity | intensity |
| Test ID | Week 0 | Week 1 | Week 2 | Week 3 | Week 4 |
| 1 | 98 | 91 | 89 | 82 | 82 |
| 2 | 97 | 64 | 55 | 43 | 38 |
| 3 | 97 | 91 | 88 | 76 | 71 |
| 4 | 97 | 89 | 81 | 72 | 67 |
| 5 | 96 | 82 | 79 | 68 | 61 |

### Example 2

In this example, the five animals from example 1 were exposed to an additional four weeks of LED light generated by the ultra bright white LEDs for 60 minutes every day. Over a period of 4 weeks, a gradual fading of the tattoo is observed as detailed in Table 2:

TABLE 2
Fading pattern of the circular tattoo without heat or a vasodilator cream
(Exposure period: 60 minutes)

| | Average | | Average | Average | Average |
|---|---|---|---|---|---|
| | color | Average color | color | color | color |
| | intensity | intensity | intensity | intensity | intensity |
| Test ID | Week 0 | Week 1 | Week 2 | Week 3 | Week 4 |
| 1 | 82 | 81 | 80 | 80 | * |
| 2 | 38 | 34 | 34 | 30 | 30 |
| 3 | 71 | 68 | 67 | 65 | 65 |
| 4 | 67 | 61 | 59 | 55 | 55 |
| 5 | 61 | 54 | 54 | 54 | 54 |

* Data could not be retrieved.

### Example 3

In this example with five rats, the tattoo was circular and square in shape. A quarantine period of one week was observed. After the completion of the quarantine period, the LED exposure was initiated on the subject tattoo. For a period of four weeks, the tattooed skin area was exposed to continuous LED light generated by LED of FIG. 2 for thirty minutes every day. Following each LED exposure, a thin layer of a vasodilator cream was applied over the tattooed skin after LED exposure. The results are detailed in Table 3:

TABLE 3
Fading pattern of the square tattoo with vasodilator cream
(Exposure period: 30 minutes).

| | Average | Average | Average | Average | |
|---|---|---|---|---|---|
| | color | color | color | color | Average color |
| | intensity | intensity | intensity | intensity | intensity |
| Test ID | Week 0 | Week 1 | Week 2 | Week 3 | Week 4 |
| 1 | 96 | 93 | 89 | 87 | 81 |
| 2 | 97 | 61 | 52 | 40 | 39 |
| 3 | 93 | 91 | 83 | 78 | 71 |
| 4 | 98 | 89 | 81 | 77 | 69 |
| 5 | 96 | 82 | 75 | 70 | 65 |

For a period of 4 weeks, the tattooed area was exposed to the continuous light energy generated by the LED for 30 minutes every day.

### Example 4

In this example, the five animals from Example 3 were exposed to an additional four weeks of LED light generated by the ultra bright white LED for 60 minutes every day. After the completion of quarantine period the LED exposure was initiated on the tattooed area. Following each exposure to the LED energy, a thin layer of the vasodilator cream was applied over the tattooed area. Over a period of 4 weeks, a gradual fading of the tattoo is observed during the entire experimental duration with the results detailed in Table 4:

TABLE 4
Fading pattern of the tattoo with vasodilator
cream (Exposure period: 60 minutes)

| | Average | Average | Average | Average | Average |
|---|---|---|---|---|---|
| | color | color | color | color | color |
| Test | intensity | intensity | intensity | intensity | intensity |
| ID | Week 0 | Week 1 | Week 2 | Week 3 | Week 4 |
| 1 | 81 | 79 | 76 | 76 | ** |
| 2 | 39 | 33 | 30 | 26 | 26 |
| 3 | 71 | 69 | 66 | 64 | 64 |
| 4 | 69 | 63 | 58 | 50 | 50 |
| 5 | 65 | 59 | 55 | 55 | 55 |

**Data could not be retrieved.

Over a period of 4 weeks, a gradual fading of the tattoo was observed during the entire experimental duration.

In order to further investigate the requirements for tattoo removal, Examples 5 to 9 were undertaken as follows:

### Example 5

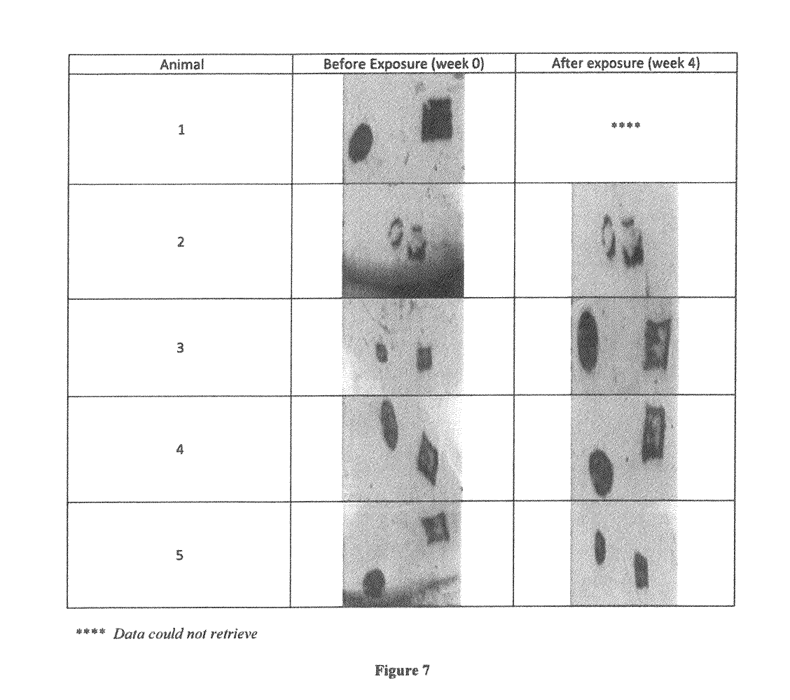

This study was conducted on two male Wistar rats. Two black tattoos were made on either side of the spine on the back of each of the animals. After the completion of quarantine period the LED exposure was initiated on the subject tattoo once a day for one hour. The LED exposure was followed by application of the vasodilator cream by gentle rubbing. Animals were monitored and fading was observed over an eight week period. The results are detailed in Tables 5(A) and 5(B) below and are shown in FIGS. 8A-8B.

TABLE 5(A)
The fading pattern of animals tattooed with black ink receiving LED
exposure once a day for one hour with application of the vasodilator cream.
Animal 1

| Duration | Week 0 | Week 1 | Week 2 | Week 3 |
|---|---|---|---|---|
| Average | 83, 84 (black) | 79, 78 (black) | 78, 78 (black) | 78, 77 (black) |
| Intensity | 85, 87 (black) | 85, 85 (black) | 84, 83 (black) | 82, 82 (black) |
| Duration | Week 4 | Week 5 | Week 6 | Week 7 |
| Average | 78, 77 (black) | 76, 77 (black) | 75, 76 (black) | 74, 76 (black) |
| Intensity | 82, 81 (black) | 80, 80 (black) | 80, 79 (black) | 79, 79 (black) |

TABLE 5(B)
The fading pattern of animals tattooed with black ink receiving LED
exposure once a day for one hour with application of the vasodilator cream.
Animal 2

| Duration | Week 0 | Week 1 | Week 2 | Week 3 |
|---|---|---|---|---|
| Average | 83, 85 (black) | 79, 77 black) | 79, 76 black) | 79, 75 (black) |
| Intensity | 87, 82 (black) | 85, 82 (black) | 83, 82 (black) | 82, 82 (black) |
| Duration | Week 4 | Week 5 | Week 6 | Week 7 |
| Average | 79, 74 (black) | 74, 74 (black) | 74, 73 (black) | 73, 72 (black) |
| Intensity | 82, 82 (black) | 81, 81 (black) | 81, 79 (black) | 79, 79 (black) |

### Example 6

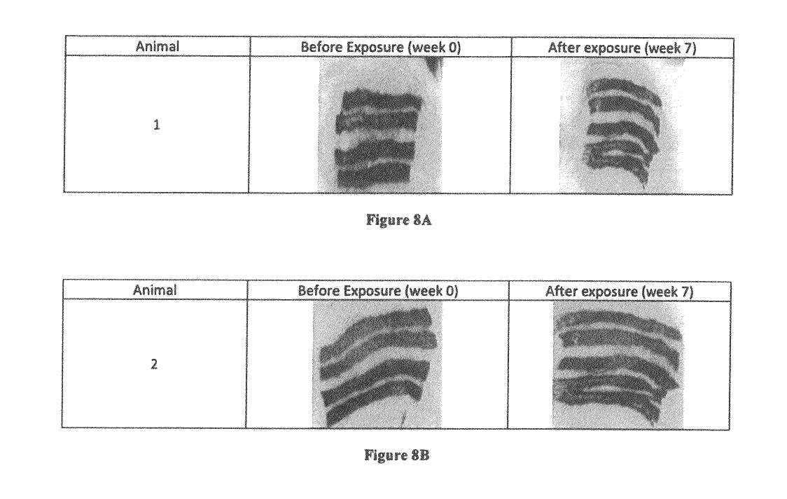

This study was conducted on two male Wistar rats. Two blue tattoos were made on either side of the spinal cord on the back of the animal. After the completion of quarantine period the LED exposure was initiated on the subject tattoo once a day for one hour. The LED exposure was followed by application of the vasodilator cream by gentle rubbing. Animals were monitored and fading observed over a period of eight weeks. The results are detailed in Tables 5(C) and 5(D) below and in FIGS. 9A and 9B.

TABLE 5(C)
The fading pattern of animals tattooed with blue
ink receiving LED exposure once a day for one
hour with application of the vasodilator cream.
Animal 1

| Duration | Week 0 | Week 1 | Week 2 | Week 3 |
|---|---|---|---|---|
| Average | 56, 59 (blue) | 55, 57 (blue) | 55, 55 (blue) | 53, 54 (blue) |
| Intensity | 63, 67 (blue) | 64, 64 (blue) | 63, 60 (blue) | 63, 60 (blue) |
| Duration | Week 4 | Week 5 | Week 6 | Week 7 |
| Average | 53, 52 (blue) | 49, 51 (blue) | 47, 50 (blue) | 45, 50 (blue) |
| Intensity | 60, 60 (blue) | 59, 59 (blue) | 58, 57 (blue) | 57, 56 (blue) |

TABLE 5(D)
The fading pattern of animals tattooed with blue
ink receiving LED exposure once a day for one
hour with application of the vasodilator cream.
Animal 2

| Duration | Week 0 | Week 1 | Week 2 | Week 3 |
|---|---|---|---|---|
| Average | 60, 67 (blue) | 59, 62 (blue) | 58, 60 (blue) | 58, 58 (blue) |
| Intensity | 64, 69 (blue) | 64, 65 (blue) | 64, 62 (blue) | 62, 62 (blue) |
| Duration | Week 4 | Week 5 | Week 6 | Week 7 |
| Average | 58, 58 (blue) | 48, 47 (blue) | 45, 47 (blue) | 44, 46 (blue) |
| Intensity | 62, 62 (blue) | 61, 62 (blue) | 61, 60 (blue) | 59, 60 (blue) |

### Example 7

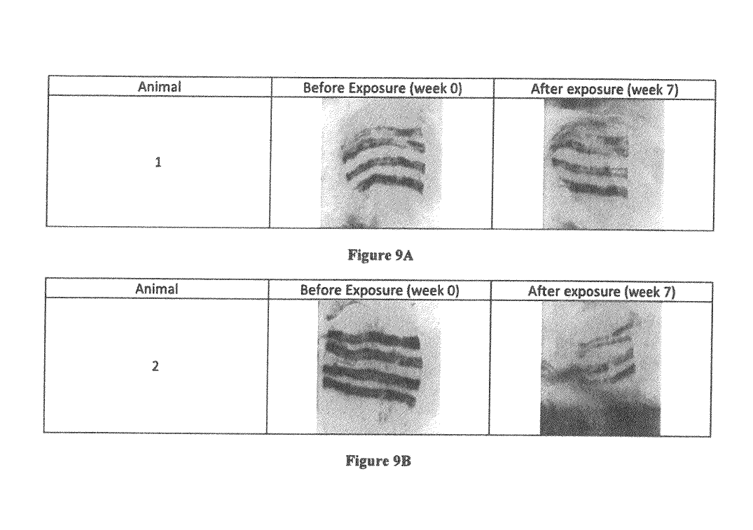

This study was conducted on two male Wistar rats. Two black tattoos were made on either side of the spine on the back of each animal. After the completion of quarantine period, the LED exposure was initiated on the subject tattoos once a day for one hour. The LED exposure was followed immediately by application of L-arginine cream by gentle rubbing. Fading was observed over a period of eight weeks. The results are detailed in Tables 6(A) and 6(B) below and FIGS. 10A and 10B.

TABLE 6(A)
The fading pattern of animals tattooed with black
ink receiving LED exposure once a day for one hour with
application of the vasodilator cream after LED exposure.
Animal 1

| Duration | Week 0 | Week 1 | Week 2 | Week 3 |
|---|---|---|---|---|
| Average | 87, 86 (black) | 76, 76 (black) | 74, 73 black) | 72, 72 (black) |
| Intensity | 87, 85 (black) | 75, 76 (black) | 75, 75 (black) | 75, 74 (black) |
| Duration | Week 4 | Week 5 | Week 6 | Week 7 |
| Average | 72, 72 (black) | 72, 71 (black) | 71, 70 (black) | 70, 70 (black) |
| Intensity | 75, 74 (black) | 74, 74 (black) | 73, 72 (black) | 73, 72 (black) |

TABLE 6(B)
The fading pattern of animals tattooed with black ink receiving LED
exposure once a day for one hour with application of a vasodilator cream.
Animal 2

| Duration | Week 0 | Week 1 | Week 2 | Week 3 |
|---|---|---|---|---|
| Average | 85, 84 (black) | 77, 78 (black) | 73, 72 (black) | 72, 71 (black) |
| Intensity | 86, 86 (black) | 80, 81 (black) | 74, 75 (black) | 73, 72 (black) |
| Duration | Week 4 | Week 5 | Week 6 | Week 7 |
| Average | 72, 71 (black) | 72, 71 (black) | 70, 70 (black) | 70, 70 (black) |
| Intensity | 72, 71 (black) | 71, 71 (black) | 68, 71 (black) | 68, 70 (black) |

### Example 8

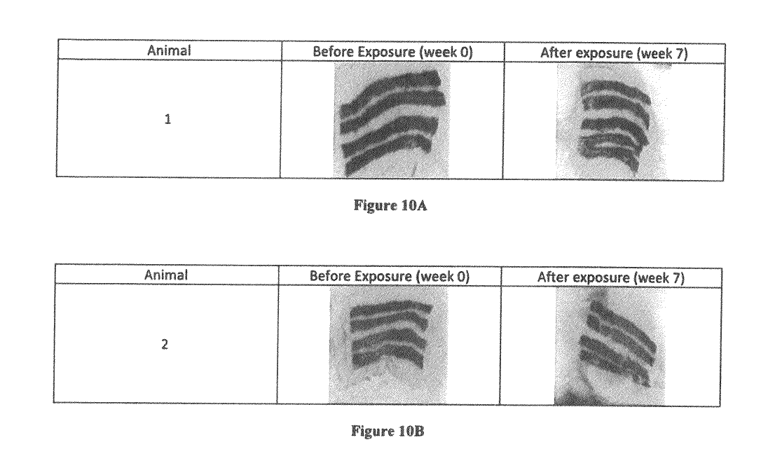

This study conducted on two male Wistar rats. Two blue tattoos were made on either side of the spinal cord on the back of each animal. After the completion of quarantine period the LED exposure was initiated on the subject tattoo once a day for one hour. The LED exposure was followed by application of the vasodilator cream by gentle rubbing. Fading was observed over a period of eight weeks. The results are detailed in Tables 6(C) and 6(D) below and FIGS. 11A and 11B.

TABLE 6(C)
The fading pattern of animals tattooed with blue
ink receiving LED exposure once a day for one
hour with application of the vasodilator cream.
Animal 1

| Duration | Week 0 | Week 1 | Week 2 | Week 3 |
|---|---|---|---|---|
| Average | 59, 61 (blue) | 57, 61 (blue) | 56, 60 (blue) | 55, 57 (blue) |
| Intensity | 62, 57 (blue) | 59, 57 (blue) | 58, 56 (blue) | 55, 54 (blue) |
| Duration | Week 4 | Week 5 | Week 6 | Week 7 |
| Average | 55, 57 (blue) | 42, 41 (blue) | 41, 40 (blue) | 40, 40 (blue) |
| Intensity | 55, 54 (blue) | 48, 45 (blue) | 48, 42 (blue) | 41, 43 (blue) |

TABLE 6(D)
The fading pattern of animals tattooed with blue
ink receiving LED exposure twice a day for one
hour with application of the vasodilator cream.
Animal 2

| Duration | Week 0 | Week 1 | Week 2 | Week 3 | Week 4 |
|---|---|---|---|---|---|
| Average | 87, 85 | 65, 63 | 63, 63 | 36, 28 | 29, 25 |
| Intensity | 86, 80 | 65, 67 | 64, 67 | 51, 59 | 30, 50 |
| Duration | Week 5 | Week 6 | Week 7 | Week 8 | Week 9 |
| Average | 27, 20 | 25, 19 | 25, 17 | 42, 46 | 42, 46 |
| Intensity | 29, 50 | 29, 50 | 28, 50 | 42, 49 | 42, 49 |

### Example 9

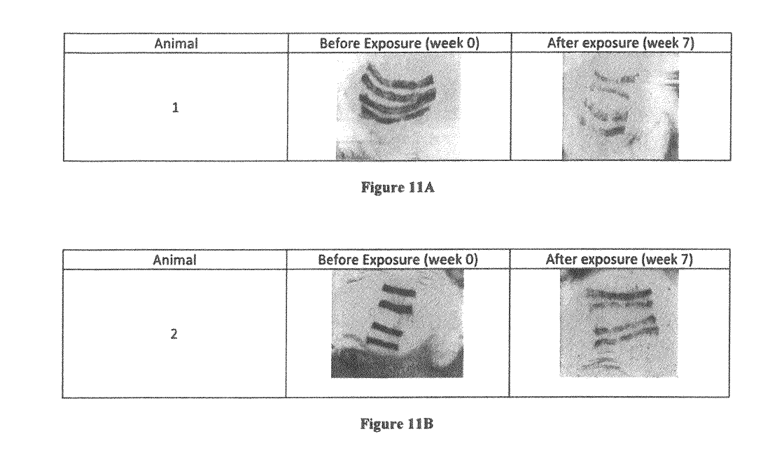

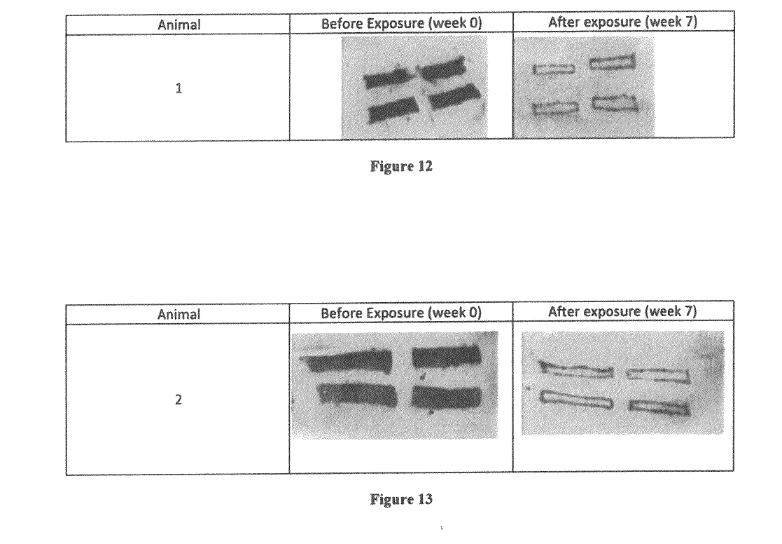

This study was conducted on two male Wistar rats. Two tattoos were made on either side of the spinal cord on the back of each animal. In Animal 1 black tattoos were applied, whereas in Animal 2 blue tattoos were applied. After the completion of a quarantine period, following application of the vasodilator cream, LED exposure was initiated on the tattooed area twice a day (i.e. two sittings per day) for one hour. Fading was observed over a period of eight weeks. Note that Table 7A below is correlated to FIG. 12 and Table 7B below is correlated to FIG. 13 respectively.

TABLE 7(A)
The fading pattern of animals tattooed with black ink receiving LED
exposure twice a day for one hour with application of the vasodilator cream.
Animal 1

| Duration | Week 0 | Week 1 | Week 2 | Week 3 |
|---|---|---|---|---|
| Average | 96, 94 (black) | 72, 71 (black) | 49, 47 black) | 36, 40 (black) |
| Intensity | 97, 96 (black) | 69, 73 (black) | 45, 46 (black) | 41, 42 (black) |
| Duration | Week 4 | Week 5 | Week 6 | Week 7 |
| Average | 42, 40 (black) | 27, 29 (black) | 25, 28 (black) | 23, 25 (black) |
| Intensity | 41, 36 (black) | 26, 24 (black) | 24, 24 (black) | 23, 24 (black) |

TABLE 7(B)
The fading pattern of animals tattooed with blue
ink receiving LED exposure twice a day for one
hour with application of the vasodilator cream.
Animal 2

| Duration | Week 0 | Week 1 | Week 2 | Week 3 |
|---|---|---|---|---|
| Average | 98, 97 (blue) | 74, 71 (blue) | 50, 48 (blue) | 37, 41 (blue) |
| Intensity | 98, 96 (blue) | 73, 74 (blue) | 47, 52 (blue) | 43, 40 (blue) |
| Duration | Week 4 | Week 5 | Week 6 | Week 7 |
| Average | 38, 40 (blue) | 24, 25 (blue) | 22, 24 (blue) | 21, 23 (blue) |
| Intensity | 42, 40 (blue) | 21, 20 (blue) | 20, 21 (blue) | 20, 20 (blue) |

TABLE 8
Initial intensity after LED exposure (LED Exposure once a day for one hour) per
Examples 5 and 6.

| | Black | | | | Blue | | |
|---|---|---|---|---|---|---|---|
| | | | Average | | | | Average |
| | | | Color | | | | Color |
| | | Average | Intensity of | | | Average | Intensity of |
| Animal | Color | Color | both | Animal | Color | Color | both |
| No. | Intensity | Intensity | animals | No. | Intensity | Intensity | animals |
| Animal 1 | 83, 84 | 84.75 | 84.50 | Animal 1 | 56, 59 | 61.25 | 63.12 |
| | 85, 87 | | | | 63, 67 | | |
| Animal 2 | 83, 85 | 84.25 | | Animal 2 | 60. 67 | 65.00 | |
| | 87, 82 | | | | 64, 69 | | |

TABLE 9
Final intensity after LED exposure (LED Exposure once a day for one hour) per
Examples 5 and 6.

| | Black | | | | Blue | | |
|---|---|---|---|---|---|---|---|
| | | | Average | | | | Average |
| | | | Color | | | | Color |
| | | Average | Intensity of | | | Average | Intensity |
| Animal | Color | Color | both | Animal | Color | Color | of both |
| No. | Intensity | Intensity | animals | No. | Intensity | Intensity | animals |
| Animal 1 | 74, 76 | 77.00 | 76.375 | Animal 1 | 45, 50 | 51.50 | 51.87 |
| | 79, 79 | | | | 57, 56 | | |
| Animal 2 | 73, 72 | 77.00 | | Animal 2 | 44, 46 | 52.25 | |
| | 79, 79 | | | | 59, 60 | | |

TABLE 10
Initial intensity after LED exposure with application of the vasodilator cream (LED
Exposure once a day for one hour) per Examples 7 and 8.

| | Black | | | | Blue | | |
|---|---|---|---|---|---|---|---|
| | | | Average | | | | Average |
| | | | Color | | | | Color |
| | | Average | Intensity | | | Average | Intensity of |
| | Color | Color | of both | | Color | Color | both |
| Animal | Intensity | Intensity | animals | Animal | Intensity | Intensity | animals |
| Animal 1 | 87, 86 | 86.25 | 85.75 | Animal 1 | 59, 61 | 59.75 | 60.62 |
| | 87, 85 | | | | 62, 57 | | |
| Animal 2 | 85, 84 | 85.25 | | Animal 2 | 60, 62 | 61.50 | |
| | 86, 86 | | | | 63, 61 | | |

TABLE 11
Final intensity after LED exposure with application of the vasodilator cream (LED
Exposure once a day for one hour) per Examples 7 and 8.

| | Black | | | | Blue | | |
|---|---|---|---|---|---|---|---|
| | | | Average | | | | Average |
| | | | Color | | | | Color |
| | | Average | Intensity of | | | Average | Intensity |
| | Color | Color | both | Animal | Color | Color | of both |
| Animal No. | Intensity | Intensity | animals | No. | Intensity | Intensity | animals |
| Animal 1 | 70, 70 | 71.25 | 70.37 | Animal 1 | 40, 40 | 41.00 | 43.25 |
| | 73, 72 | | | | 41, 43 | | |
| Animal 2 | 70, 70 | 69.50 | | Animal 2 | 40, 40 | 45.50 | |
| | 68, 70 | | | | 51, 51 | | |

TABLE 12
Comparison of average initial and final
intensities of Examples 7 and 8.

| | | | Change = |
|---|---|---|---|
| Animal No. | Final | Initial | Initial − Final |
| Black | 70.37 | 85.75 | 15.38 |
| Blue | 43.25 | 60.62 | 17.37 |

TABLE 13
Comparison of average initial and final intensities of Example 9.

| | | | Change = |
|---|---|---|---|
| Animal No. | Final | Initial | Initial − Final |
| Black | 30.00 | 84.50 | 54.50 |
| Blue | 44.75 | 87.25 | 42.50 |

TABLE 14
Comparison of average initial intensities of Example 10.

| | Black | | | | Blue | | |
|---|---|---|---|---|---|---|---|
| | | | Average | | | | Average |
| | | | Color | | | | Color |
| | | Average | Intensity | | | Average | Intensity |
| | Color | Color | of both | | Color | Color | of both |
| Animal | Intensity | Intensity | animals | Animal | Intensity | Intensity | animals |
| Animal 1 | 96, 94 | 95.75 | 95.75 | Animal 2 | 98, 97 | 97.25 | 97.25 |
| | 97, 96 | | | | 98, 96 | | |

TABLE 15
Comparison of average final intensities of Example 10

| | Black | | | | Blue | | |
|---|---|---|---|---|---|---|---|
| | | | Average | | | | Average |
| | | | Color | | | | Color |
| | | Average | Intensity | | | Average | Intensity |
| | Color | Color | of both | | Color | Color | of both |
| Animal | Intensity | Intensity | animals | Animal | Intensity | Intensity | animals |
| Animal 1 | 23, 25 | 23.75 | 23.75 | Animal 2 | 21, 23 | 21.00 | 21.00 |
| | 23, 25 | | | | 20, 20 | | |

TABLE 16
Comparison of average initial and final intensities of Example 10

| | | | Change = |
|---|---|---|---|
| Animal No. | Final | Initial | Initial − Final |
| Black | 23.75 | 95.75 | 72.00 |
| Blue | 21.00 | 97.25 | 76.25 |

### Theory

For the LED light produced to be beneficial for photobiomodulation (i.e. alteration of cellular function) or in the present application for removal of tattoos, LEDs with sufficient energy output must be utilized. The present tattoo removal system uses ultra bright LEDs in accordance with the spectrum and intensively shown in FIG. 1.

In general, it is well known that light demonstrates both a particle and wave nature. In its particle nature, light consists of packets of energy called photons. At any given wavelength, all photons have identical energy given by the equation:

Energy=Planck's Constant×Speed of Light/Wavelength.

The brightness of light depends upon its photon density. The brighter the light, the greater will be its photon density since each photon at a given wavelength has the same energy. The greater the photon density, the greater will be the energy content of the light. Thus, use of ultra bright LEDs leads to a higher level of energy in the light beam. The greater the light intensity (i.e. higher millicandela values), the greater will be the energy content of the light.

### Operation

In operation, the tattoo removal system and method of use in accordance with the present invention is carried out by irradiating the tattooed skin with continuous (i.e. non-pulsed) irradiation in a predetermined range of wavelengths as previously described in the orange red and red wavelengths. The LED light energy generated by an LED is capable of treating large skin surface areas.

The time that the tattooed skin area is exposed to the light of the LED is at least five minutes per day. The LED light of FIG. 1 penetrates to the deep inside of the tattooed skin and facilitates removal of the tattoo ink from the affected area.

One particular vasodilator that was used had the following composition.

| Sr. | | |
|---|---|---|
| No | Ingredient | % content |
| 1 | Glyceryl monostearate | 10% |
| 2 | Petrolatum | 10% |
| 3 | Light mineral oil | 17.5% |
| 4 | Lanolin base | 15% |
| 5 | Bees wax | 7.5% |
| 6 | Propyl paraben | 0.15% |
| 7 | Methyl Paraben | 0.15% |
| 8 | Sodium Chloride | 5% |
| 9 | Arginine | 15% |
| 10 | Water | 20% |

(Note: the percentages are approximate and add to more than 100%)

Using the present tattoo removal system and method, persons having tattooed skin can a person or a medical person or themselves to administer a daily dosage of ultra bright LED energy on the affected skin area on their own without the services and related expense of a physician or other highly trained healthcare professional.

In accordance with the present method of administering treatment, the tattooed skin area is exposed to LED energy about for 30 minutes per sitting. A vasodilator such as the vasodilator cream or heated water as vehicle is applied to the subject tattoo after the LED exposure.

The distance between the LED and the tattooed skin area is maintained at about approximately 10 centimeters. The LED is adjusted vertically above the tattooed skin area so that optimum exposure to LED energy is ensured. During each week of treatment, excess hair may be removed by shaving or hair removing cream if desired. A gradual fading of tattoos will be observed over a period of nine weeks of treatment.

The present method has proven to be effective even for fading of tattoos created using different colors of ink. To provide an objective standard for measuring tattoo removal effectiveness, the intensity of the tattoos, the basic colors of cyan (i.e. blue), magenta (i.e. red), yellow, and black present in the tattooed skin are measured for intensity.

The percentage intensity of the tattooed skin that is exposed to the treatment according to the present invention is usefully measured by a commercially available software program sold under the tradename, Colorpic software. The average percentage intensity is measured by scanning about ten arbitrary reference points on each tattooed skin area to be evaluated.

The wavelength of the ultra bright LEDs that are used in the present system has a principal peak at 640 nm to 700 nm with the spectrum substantiality of FIG. 1 in wavelength. In that range there appears to be the most energy imparted to the tattoo and there is little light absorption by either melanin, a skin coloring agent, or hemoglobin, so absorption by the dye is maximized. It understood that 640 nm-700 nm the red wavelengths yield the best results.

Heat is generated when the light is absorbed by the irradiated tattoo ink molecules thereby increasing the molecular motion. As configured herein, the LED of this irradiation will not generate tissue-damaging heat on the skin but will generate sufficient energy within tattoo dyes. Therefore, little to no heat is generated on the surface of the skin when using the present method, but the LED light penetrates through the epidermis into the dermis where the tattoo ink is disposed and the light frequencies are absorbed by tattoo dye molecules causing increased molecular motion and bond deformation.

Additionally the application of a vasodilator cream or external heat source will increase the degree of tattoo removal. The cream used in the experiments is a chemical vasodilator that causes enlargement of blood vessels. The application of the cream to the skin causes increased blood flow to the tattoo area. In addition, the vasodilator, the cream, increases the concentration of macrophage cells in the blood and also strengthens the immune system response. The increased molecular motion and chemical bond deformation within the tattoo ink combined with increased blood flow to the tattoo area stimulates the subject's immune response to metabolize the molecular by-products of tattoo ink.

It will be understood that the hereinabove described embodiments of the present invention are intended to be illustrative of some of the applications or principles involved therein. Various modifications may be made by the skilled person without departing from the true spirit of the invention.

Having described preferred embodiments of my invention, what I desire to secure by U.S. Letters Patent is:

1. A tattoo removal device comprising:
an LED with a light spectrum that has a principal peak at about 640 nm-700 nm which when focused on a tattoo will cause the tattoo to fade in intensity.

2. the LED of claim 1 having the wavelength at about 640 nm-700 nm, wherein said LED is configured to irradiate a tattooed skin region with continuous LED energy to lessen the intensity of the tattoo;

3. A method for removing tattoos comprising the steps of:
positioning a tattoo removal device including an LED at an efficacious distance of about 10 cm, from said tattooed skill region, to give the best medical efficacy and
Irradiating an area having a tattoo thereon with an LED with a light spectrum having a wavelength at about 640 nm-700 nm.
exposing said tattooed skin region to continuous LED energy without pulsing for an efficacious timed interval.

4. The method of claim 3 wherein the step of applying further includes the step of applying a vasodilator to the tattoo area after irradiating the tattoo area with LED light in the red wavelengths.

5. The method of claim 3 wherein vasodilator is L-arginine.

6. The method of claim 3 wherein the LED has a power of at least 50 W.

7. The method of claim 3 wherein a heating source is used on the tattoo area after irradiating the area with LED light.

8. The method of claim 6 wherein the heating source is an electrical or chemical heating source.

9. A method for removing tattoos comprising the steps of:
Irradiating an area having a tattoo thereon with an LED with a light spectrum having a wavelength at about 640-700 nm
positioning a tattoo removal device including an LED having at an efficacious distance of less than 10 cm, from said tattooed skin region, to give the best medical effect and
exposing said tattooed skin region to continuous LED energy without pulsing for an efficacious time interval.

10. The method of claim 3 wherein the step of applying further includes the step of applying a vasodilator to the tattoo area after irradiating the tattoo area with LED light.

11. the method of claim 3 wherein the vasodilator is L-arginine.

12. The method of claim 3 wherein the LED has a power of at least 50 W-3000 joules per minute.

13. The method of claim 3 wherein a heating source is used on the tattoo area after irradiating the area with LED light.

14. A method for removing tattoos comprising step of:
positioning a tattoo removal device including an LED having a spectrum having a principal peak at 640-700 nm, for apply energy to a tattooed skin region;

15. The method of claim 10 further including the step of applying a vasodilator cream to said tattooed skin region.

16. The method of claim 10 wherein the step of irradiating further including the step of exposing said tattooed skin region to continuous LED light energy for an efficacious timed interval exceeding five minutes.

17. The LED of claim 1 having LED light in the red or orange-red spectrum.

18. The LED of claim 1 having LED light in the warm light spectrum.

19. The method of claim 3 wherein the step of irradiating further includes the step of exposing said tattooed skin region to continuous LED light energy for an efficacious timed interval exceeding five minutes.

20. The method of claim 8 wherein the step of irradiating further includes the step of exposing said tattooed skin region to continuous LED light energy for an efficacious timed interval of 1 hour per day for one month.
